(19) United States
(12) Patent Application Publication
(10) Pub. No.: US 2026/0151427 A1
(43) Pub. Date: Jun. 4, 2026

(54) LANTHANUM COMPOSITIONS FOR TREATING CANCER

(71) Applicant: Zetagen Therapeutics, Inc., Syracuse, NY (US)

(72) Inventor: Bryan S. Margulies, Liverpool, NY (US)

(21) Appl. No.: 19/405,165

(22) Filed: Dec. 1, 2025

Related U.S. Application Data
(60) Provisional application No. 63/726,990, filed on Dec. 2, 2024.

(51) Int. Cl. A61K 33/244; A61K 31/485; A61K 45/06; A61K 47/36

(52) U.S. Cl. CPC A61K 33/244; A61K 31/485; A61K 45/06; A61K 47/36

(57) ABSTRACT

Described herein are compositions comprising a lanthanum compound and methods of treating cancer with the composition.

### RELATED APPLICATIONS

The present application claims priority to U.S. provisional application No. 63/726,990 filed Dec. 2, 2024, which is incorporated herein by reference in its entirety.

### SEQUENCE LISTING

The instant application contains a Sequence Listing which has been submitted electronically in XML format and is hereby incorporated by reference in its entirety. Said XML copy, created on Jan. 19, 2026, is named 118922-0354_SL.xml and is 8,824 bytes in size.

### FIELD

The present invention relates in general to pharmaceutical formulations, their uses for treating oncologic diseases and their methods of making and more specifically to controlled release pharmaceutical formulations, their uses, which may involve minimally invasive administration, as well as their methods of making.

### SUMMARY

One embodiment is a composition for treating cancer, comprising a lanthanum compound and a pharmaceutically acceptable carrier. In some embodiments, the lanthanum compound may be an inorganic lanthanum salt. In some embodiments, the lanthanum compound may include lanthanum (III) carbonate, lanthanum (III) sulfate, lanthanum (III) nitrate, lanthanum (III) oxide, lanthanum (III) chloride, and lanthanum (III) acetate. functional derivative thereof. In one aspect wherein the lanthanum is included in an amount of about 0.1% to 85% (w/w). In one aspect the lanthanum is included at a concentration between 10-nM and 10-mM.

Another embodiment is a method of treating a cancer comprising administering to a subject in need thereof an effective amount of a lanthanum compound.

In one aspect, the method comprises administering to a subject in need thereof a composition comprising a lanthanum compound contained in a suitable carrier or vehicle.

In some embodiments, an additional therapeutic agent is included in the compositions and therapeutic methods described herein. The additional therapeutic agent may be administered in the same composition or co-administered. In some embodiments, the additional therapeutic agent is applied concurrently or consecutively to the composition comprising a lanthanum compound. In some embodiments the additional therapeutic agent is an anti-cancer therapeutic agent. In some embodiments, the anti-cancer therapeutic agent is a chemotherapeutic agent, a hormone therapy, a CDK inhibitor, or an OGFR antagonist.

### FIGURES

FIGS. 1A-1B. Schematic depicting the mechanism by which lanthanum has a anti-tumorigenic effect. FIG. 1A shows a standard cell, which shows calcium moving through calcium channels. FIG. 1B shows a cell contacted with lanthanum, which moves through calcium channels, and where calcium does not move through calcium channels. FIG. 1B also shows the cell contacted with N-allyl noroxymorphone, which may act as an OGFR antagonist.

FIG. 2. Images showing decreased metabolic activity in cultured BT474 breast cancer cells.

FIG. 3. IC50 calculated for the MCF7 breast cancer cells and the BT474 breast cancer cells. The MCF7 IC50 is 237.6-μg and the BT474 IC50 is 217.5-μg. These data show a high level of sensitivity to lanthanum carbonate treatment.

FIGS. 4A-4B. The effects of lanthanum carbonate hydrogel on cell proliferation and cell death using the MTT (FIG. 4A) and LDH (FIG. 4B) assays.

### DETAILED DESCRIPTION

As used in this disclosure and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

All numerical designations, e.g., pH, temperature, time, concentration, amounts, and molecular weight, including ranges, are approximations which are varied (+) or (−) by 10%, 1%, or 0.1%, as appropriate. It is to be understood, although not always explicitly stated, that all numerical designations may be preceded by the term “about.” It is also to be understood, although not always explicitly stated, that the reagents described herein are merely exemplary and that equivalents of such are known in the art.

The term “comprising” or “comprises” is intended to mean that the compositions and methods include the recited elements, but do not exclude others. “Consisting essentially of,” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other the ingredients and substantial method steps recited by the claims. Embodiments defined by each of these transition terms are within the scope of this invention.

As used here, the term “antagonist” is used interchangeably with “inhibitor” and refers to a substrate that blocks or suppresses the activity, function, effect, or expression of a target. In some embodiments, the target is a compound, a protein, a gene, a cell, or an agent. As used herein, the term “expression” refers to the amount a living cell produces of a target. In some embodiments, the inhibitor suppresses expression of a target gene or protein. In some embodiments, the inhibitor includes a compound that prevents binding of another molecule to an enzyme or molecular pump. In some embodiments, the inhibitor is a compound that causes downregulation of the enzyme. In some embodiments, the inhibitor can be a competing or non-competing inhibitor.

The term “administering” as used herein includes prescribing for administration as well as actually administering, and includes physically administering by the subject being treated or by another.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

As used herein “subject,” “patient,” or “individual” refers to any subject, patient, or individual, and the terms are used interchangeably herein. In this regard, the terms “subject,” “patient,” and “individual” includes mammals, and, in particular humans. When used in conjunction with “in need thereof,” the term “subject,” “patient,” or “individual” intends any subject, patient, or individual having or at risk for a specified symptom or disorder.

As used herein, the phrase “therapeutically effective” or “effective” in context of a “dose” or “amount” means a dose or amount that provides the specific pharmacological effect for which the compound or compounds are being administered. It is emphasized that a therapeutically effective amount will not always be effective in achieving the intended effect in a given subject, even though such dose is deemed to be a therapeutically effective amount by those of skill in the art. For convenience only, exemplary dosages are provided herein. Those skilled in the art can adjust such amounts in accordance with the methods disclosed herein to treat a specific subject suffering from a specified symptom or disorder. The therapeutically effective amount may vary based on the route of administration and dosage form.

The term “treating” or “treatment” covers the treatment of a cancer described herein, in a subject, such as a human, and includes (i) inhibiting a cancer, i.e., arresting its development; (ii) relieving a cancer or disorder, i.e., causing regression of the cancer; (iii) slowing progression of the cancer; and/or (iv) inhibiting, relieving, or slowing progression of one or more symptoms of the cancer. For example, treatment of a cancer includes, but is not limited to, elimination of the cancer or the condition caused by the cancer, remission of the tumor, inhibition of the cancer, or reduction or elimination of at least one symptom of the tumor.

The term “analog” refers to a compound in which one or more individual atoms or functional groups have been replaced, either with a different atom or a different functional group, generally giving rise to a compound with similar properties. In some aspect, the analog refers to a structure that is similar to another but differs in one or two components.

The term “derivative” refers to a compound that is formed from a similar beginning compound by attaching another molecule or atom to the beginning compound. Further, derivatives, according to the invention, encompass one or more compounds formed from a precursor compound through addition of one or more atoms or molecules or through combining two or more precursor compounds.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. As used herein, the terms “composition” and “formulation” are used interchangeably.

The disclosure relates to a composition comprising lanthanum compounds. The composition may be a controlled release formulation. The composition may also include at least one pharmaceutically acceptable carrier. Preferably, the pharmaceutically acceptable carrier is a hydrogel. The composition may an additional therapeutic agent. The additional therapeutic agent may be an OGFR antagonist.

The disclosure also relates to therapeutic methods of using formulations of lanthanum compounds to treat cancers. The cancer

### Lanthanum Compounds

By “lanthanum compound” is meant any molecule that comprises lanthanum. Lanthanum is a lanthanide metal. In the methods and compositions described herein, the lanthanum compound may be used to treat cancer.

In some embodiments, the lanthanum compound may be an inorganic lanthanum salt or a derivative thereof. In some embodiments, the lanthanum compound may include lanthanum (III) carbonate, lanthanum (III) sulfate, lanthanum (III) nitrate, lanthanum (III) oxide, lanthanum (III) chloride, and lanthanum (III) acetate

In some embodiments, the lanthanum compound is present in the composition in an amount of 0.1% to 85% (w/w). In some embodiments the lanthanum compound is present in the composition at a concentration between 10-nM and 10-mM.

### Lanthanum Salts

### Lanthanum Modifications of Protein and Enzyme Activity:

Calmodulin (CaM):

-CaM is a ubiquitous Ca²⁺-binding protein which mediates intracellular responses to Ca²⁺ fluxes. It does this by interacting with specific receptor proteins in a Ca²⁺-dependent manner. It is a highly acidic protein of pH 4. It is a dumbbell-shaped molecule, with two globular lobes connected by a long, exposed alpha-helix. Each lobe binds two Ca²⁺ ions through the helix-loop-helix structure that is typical of “E-F” handed proteins. Upon binding Ca²⁺, the protein undergoes marked conformational changes, exposing a hydrophobic domain. La³⁺ can substitute for Ca²⁺ in the Ca²⁺-dependent attachment of CaM to cell membranes. At sub-optimal concentrations, Ca²⁺ and La³⁺ have an additive effect. However, nanomolar concentrations of La³⁺ inhibit the CaM-regulated guanylate cyclase by dissociating CaM from the enzyme. High concentrations of La³⁺ usually inhibit CaM-mediated processes. Indeed, treatment with millimolar concentrations of La³⁺ is one technique used to dissociate CaM from enzymes and membranes.

Ca²⁺/Mg²⁺-ATPase:

Cells need to regulate very closely their cytosolic Ca²⁺ concentrations, usually maintaining them at sub-micromolar levels. This is achieved despite the presence of an extracellular milieu where the Ca²⁺ concentration is often in the millimolar range. Such regulation is aided by specialized Ca²⁺ “pumps” which harness the energy released by hydrolysis of the terminal phosphodiester bond of ATP to transport Ca²⁺ across cellular membranes. These enzymes hence show ATPase activity; as they require both Ca²⁺ and Mg²⁺, they are referred to as Ca²⁺/Mg²⁺-ATPases. Such enzymes are associated with cell membranes and may transport Ca²⁺ across the limiting membranes of intracellular organelles, such as mitochondria and the endoplasmic reticulum, or across the plasma membrane, thus ejecting Ca²⁺ from the cell altogether. There also exist Mg²⁺-ATPases, which do not require Ca²⁺, and Na⁺/K⁺-ATPases, which transport monovalent cations. Many of the physiological effects of lanthanides reflect their influence on cellular calcium homeostasis. The best studied example is the Ca²⁺/Mg²⁺-ATPase of the sarcoplasmic reticulum (SR), an intracellular, membranous organelle of skeletal muscle cells, corresponding to the endoplasmic reticulum (ER) of non-muscle cells. During muscle contraction, the sarcoplasmic reticulum releases Ca²⁺ which triggers the interaction between the thick and thin myofilaments. During muscle relaxation, Ca²⁺ is transported, against a Ca²⁺ gradient, across the sarcoplasmic reticulum. It is this second step which is the energy-dependent process catalyzed by Ca²⁺/Mg²⁺-ATPase. Two moles of Ca²⁺ are transported per mole of ATP hydrolyzed. La³⁺ has been shown to inhibit the Mg²⁺-ATPase and Na⁺/K⁺-ATPase in rat heart sarcolemma and erythrocytes. It is possible to suppress completely Ca²⁺ efflux from erythrocytes at La³⁺ concentrations where the total ATPase is only reduced by 50%.

### Kinases:

Phosphoglycerate kinase catalyzes the reversible phosphorylation of 3-phosphoglycerate by ATP. Lanthanum inhibits the forward reaction by forming La-ATP-complex, which competes with Mg-ATP as substrates for the reaction. The K-values for La-ATP is about 0.04-mM, compared with a K-value of 0.073-mM for Mg-ATP. No conformational change in the La-ATP complex upon binding to the enzyme has been detected. La-ATP complexes also inhibit hexokinase, the enzyme which catalyzes the reversible phosphorylation of glucose. Inhibition is competitive with respect to Mg-ATP. When free Mg²⁺ is added, enzymic activity reappeared due to the competitive displacement of La³⁺ ions from ATP. Pyruvate kinase reversibly catalyzes the phosphorylation of ADP via phosphoenolpyruvate. It is strongly inhibited by lanthanum. This enzyme requires Mn²⁺ or Mg2+ for activity. Competition experiments showed that Mn²⁺, Mg²⁺, Ca²⁺, and La³⁺ can bind to pyruvate kinase. The K-value for the interaction of La-ATP with pyruvate kinase was estimated as 13-μM. La³⁺ may inhibit enzymic activity by blocking a conformational change induced by the binding of a substrate. Myosin light chain kinase is regulated by Ca²⁺-calmodulin. At a concentration of 100-μM, La3+ is able to support over 60% of the enzymatic activity produced by the same concentration of Ca²⁺. Protein kinase C requires both phospholipid and Ca²⁺. La³⁺ at 10- to 300-μM concentrations can replace Ca²⁺ in supporting protein kinase C activity. La³⁺ at concentrations of 100-μM have been shown to slightly inhibited cGMP and cAMP dependent protein kinases.

### Alkaline Phosphatase:

The alkaline phosphatase contains four Zn²⁺ ions and requires the binding of one Mg²⁺ to become enzymatically active. La³⁺ can occupy the Mg²⁺ site, with K_{d} of 0.16-μM, which results in protein conformational changes that would effect enzyme activity.

### Transient Receptor Potential (TRP) Channel Receptors:

Some transient receptor potential (TRP) channels have been shown to be permeable to trace metal ions, and it has been demonstrated in some cases that TRP channels are important for the physiological uptake of trace metal ions. In mammals, the TRP superfamily is subdivided into six families named TRPA, TRPC, TRPM, TRPML, TRPP and TRPV, each family comprising up to eight members. TRP channels conduct Ca²⁺, with most of them are located in the plasma membrane, but some are found both in the plasma membrane and in intracellular membranes (e.g. TRPM1, TRPM2, TRPM7, TRPM8, TRPC3, TRPV1 and TRPV4) and still others are exclusively found on intracellular membranes (e.g. TRPML channels).

Lanthanum has been used to inhibit most mammalian TRP channels. Lanthanum also potentiates signaling is a smaller number of TRP channels that include TRPC1, TRPC4, and TRPC5 channels. This stimulating effect is seen for concentrations of La³⁺ ranging from 1- to 1,000-μM. At still higher concentrations (5 mM), La³⁺ blocks TRPC5 current. Extracellular Ca²⁺ ions are also capable of stimulating TRPC5 channels (concentration range 2- to 20-mM). At 20-mM extracellular Ca²⁺, La³⁺ ions were found not to have an additional potentiating effect, indicating that Ca²⁺ competes with La³⁺ for the same binding site. Two negatively charged glutamate residues (Glu543 and Glu595), close to the extracellular mouth of the pore, are controlling the positive regulatory processes. The same amino acids are also important for the activation of TRPC5 channels by Ca²⁺ ions.

TRPV1 proteins also are activated by lanthanum. The application of La³⁺ at concentrations of 10- to 1,000-μM elicits currents, indicating direct activation of TRPV1 channels. In addition, at concentrations lower than 100-μM, La³⁺ potentiates TRPV1 activity induced by heat, acid, and capsaicin. The La³⁺-dependent potentiation seems to involve two glutamate residues at position 600 and 648 of the TRPV1. Human TRPV6 channels are modulated by low concentrations of extracellular La³⁺, which increases Ca²⁺ currents slightly (<50%); whereas at higher concentrations, La³⁺ depress hTRPV6-dependent Ca²⁺ currents. TRPV5 channels are highly sensitive to lanthanum and are inhibited by La³⁺ with an IC50 of 4.6-μM.

TRPA1 channels are inhibited by La³⁺, with an IC50 value that strongly depends on the concentration of extracellular Ca²⁺. At approximately physiological Ca²⁺ concentrations (2-mM), the IC50 values are 300-μM for La³⁺. At 20-μM Ca²⁺, however, the lanthanum is much more potent, with an IC50 of 54-μM.

TRPML1 channels are inhibited by 100-μM of La3+ while TRPML3 channels are much more sensitive with an IC50 of 15-μM. Several studies found that TRPM4 channels are inhibited by La³⁺. Approximately 50% inhibition by 30-μM La³⁺ was proposed when these channels were activated by mechanical stretch. The situation is similar for TRPM5, which was inhibited by 100-M La³⁺. TRPM7 channels were found to be insensitive to low concentrations of La³⁺, but 10-mM blocked inward and outward currents completely. La³⁺ ions at 2- and 10-mM were found to block inward currents through TRPM7 completely, while outward currents were only partially inhibited even at these very high concentrations.

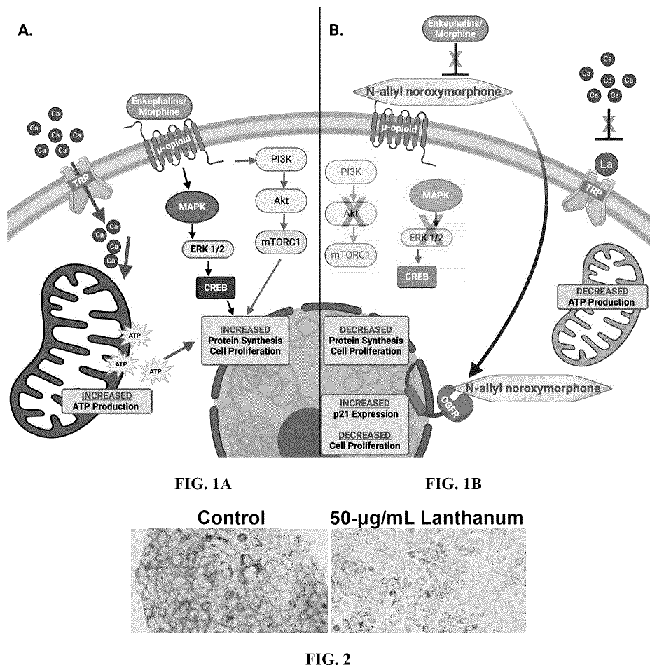

TRP channels and the μ-opioid receptor collaborate to drive cell division (FIGS. 1A-1B). TRP receptors also mediate cellular functions that include the regulation of intracellular signaling proteins (e.g., calmodulin) and mitochondria. The μ-opioid-receptor (MOR) mediates cellular function through activation of MAPK or PI3K signaling (FIGS. 1A-1B). Protein synthesis and cell proliferation are increased after opioid receptor binding of an opioid drug (e.g., morphine) or one of the native enkephalin proteins (e.g., met-5 enkephalin). Lanthanum (La) can substitute for calcium to block calcium channels. Calcium is required for cell proliferation via the increased need for ATP or through calmodulin-dependent mechanisms. Loss of calcium influx via lanthanum can result in decreased mitochondrial ATP production and decreased cell proliferation.

### Lanthanum Effects on Nucleic Acids:

The affinities of lanthanum for polynucleotides exceed their affinities for individual nucleotides, with tRNA providing stronger ligands than DNA. Urea increases the lanthanum binding to poly(G) by preventing the formation of four-stranded helices. This is consistent with the general observation that the greater the degree of secondary structure in a nucleic acid or polynucleotide, the lower is its ability to bind lanthanum. Further, the affinity of the double stranded forms exceeds that of the single stranded forms, which suggests a preferential binding of La³⁺, at low concentration, to the double stranded G-C regions. Further, thermally denaturing DNA increases La³⁺ binding and increases Terbium (Tb), another lanthanide that is luminescent, luminescence. This latter property of Terbium can be used to monitor rates of DNA reannealing, to detect subtle changes in conformation that lead to local melting of the helix, and to detect single stranded DNA bands on gels after electrophoresis. Supporting evidence comes from the observation that treatment of commercial DNA preparations with nucleases greatly reduces their ability to enhance Tb³⁺ luminescence. Titration studies suggest that lanthanum binds the DNA phosphates moieties.

### Lanthanum Activity in Mitochondria:

Lanthanum inhibits both energy-dependent transport and energy-independent binding of Ca²⁺ by mitochondria. In so doing, lanthanum inhibits Ca²⁺-dependent activation of respiration, oxidation of cytochromes, increases in intra mitochondrial pH, swelling, and increases in acetyl-coenzyme A permeability, without affecting oxidative phosphorylation or monovalent cation accumulation. Ca²⁺-dependent oxygen consumption is strongly inhibited by the addition of 3.1-μM La³⁺. La³⁺ transport has also been invoked to explain the enhanced efflux of Ca²⁺ from the mitochondrion in response to the addition of La³⁺ ions. The Ca²⁺ transporter has several Ca²⁺-binding sites; however, the binding of La³⁺ to only one of these sites inhibits Ca²⁺ transport. High-affinity binding sites may be the Ca²⁺ transporter of the inner mitochondrial membrane, which has a Kₐ for La³⁺ of 0.83-μM. The slow decay of observed in luminescence studies occurred by transfer of energy to suitable chromophores, probably the heme groups of the cytochrome proteins. All the cytochromes, apart from cytochrome C, are buried in the membrane of mitochondria core, 20- to 50-Å from the surface. Measurements have suggested a positive μM local surface potential in intact mitochondria but a negative potential in sub-mitochondrial particles. This was interpreted as reflecting two different types of binding sites, possibly the matrix and cytoplasmic sides of the Ca²⁺ transporter. La³⁺ ions produced a 50% inhibition at 0.2-nmol/mg for the Ca²⁺ transporter.

### Lanthanum Activity in Endoplasmic Reticulum:

Millimolar concentrations of La3+ are inhibitory in the endoplasmic reticulum (ER). La³⁺ also binds to the low-affinity Ca²⁺ sites with a Kₐ value of 6.5-μM, whereas the Kₐ, for Ca²⁺ is 32-μM. Binding of La³⁺ to the ER is greater in the presence of adenosine triphosphate (ATP). The ability of La³⁺ ions to compete for Ca²⁺ on the ER is reflected in the high concentrations that need to be added before inhibition of Ca²⁺ uptake is observed in the ER. At such concentrations, microsomal Ca²⁺-ATPase is also inhibited. Inhibition of Ca²⁺ uptake has been reported to be non-competitive, with 50% of the inhibition occurring with 125-μM La³⁺. In addition to using the Ca²⁺-ATPase-driven reaction, microsomes can accumulate Ca²⁺ via the Na—Ca²⁺ ion exchanger.

### Anti-Cancer Therapies

The present disclosure contemplates the use of the lanthanum compounds described herein in combination with one or more additional therapies useful in the treatment of cancer.

In some embodiments, one or more of the additional therapies is a therapeutic agent. Exemplary therapeutic agents include chemotherapeutic agents, radiopharmaceuticals, hormone therapies, CDK inhibitors, epigenetic modulators, ATP-adenosine axis-targeting agents, targeted therapies, signal transduction inhibitors, RAS signaling inhibitors, PI3K inhibitors, arginase inhibitors, HIF inhibitors, AXL inhibitors, PAK4 inhibitors, immunotherapeutic agents, cellular therapies, gene therapies, immune checkpoint inhibitors, and agonists of stimulatory or co-stimulatory immune checkpoints.

In some embodiments, the additional anti-cancer therapeutic agent is a chemotherapeutic agent. Examples of chemotherapeutic agents include, but are not limited to: alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, pomalidomide, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pemetrexed, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab paclitaxel, and docetaxel; chlorambucil; gemcitabine; 6-thioguanine; mercaptopurine; methotrexate; platinum and platinum coordination complexes such as cisplatin, carboplatin and oxaliplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT 11; proteasome inhibitors such as bortezomib, carfilzomib and ixazomib; topoisomerase inhibitors such as irinotecan, topotecan, etoposide, mitoxantrone, teniposide; difluoromethylornithine (DMFO); retinoic acid; esperamicins; capecitabine; anthracyclines and pharmaceutically acceptable salts, acids or derivatives of any of the above.

In some embodiments, the additional anti-cancer therapeutic agent is a hormone therapy. Hormone therapies act to regulate or inhibit hormonal action on tumors. Examples of hormone therapies include, but are not limited to: selective estrogen receptor degraders such asfulvestrant, giredestrant, SAR439859, RG6171, AZD9833, rintodestrant, ZN-c5, LSZ102, D-0502, LY3484356, SHR9549; selective estrogen receptor modulators such as tamoxifen, raloxifene, 4-hydroxytamoxifen, trioxifene, keoxifene, toremifene; aromatase inhibitors such asanastrozole, exemestane, letrozole and other aromatase inhibiting 4(5)-imidazoles; gonadotropin-releasing hormone agonists such as nafarelin, triptorelin, goserelin; gonadotropin-releasing hormone antagonists such as degarelix; antiandrogens such as abiraterone, enzalutamide, apalutamide, darolutamide, flutamide, nilutamide, bicalutamide, leuprolide; 5α-reductase inhibitors such as finasteride, dutasteride; and the like.

In some embodiments, the additional anti-cancer therapeutic agent is a cyclin-dependent kinase (CDK) inhibitor. Examples of CDK inhibitors include but are not limited to: flavopiridol, roscovitine, RO-3306, dinaciclib, milciclib, palbociclib, ribociclib, abemaciclib, BS-181, DRB, meriolin 3, variolin B, meridianin E, nortopsentins, AZD5438, roniciclib, SNS-032, H-Ala-Ala-Abu-Arg-Arg-Leu-Ile-pFPhe-NH2 (SEQ ID NO: 1), H-His-Ala-Lys-Arg-Arg-Leu-Ile-Phe-NH2 (SEQ ID NO: 2), MM-D37K, sorafenib, K03861, PD184352 (CI-1040), 8-anilino-1-naphthalene sulfonate (ANS), THZ531, THZ1, SY-1365, and E9.

In some embodiments, the additional anti-cancer therapeutic agent is an OGFR (opioid growth factor receptor) antagonist).

### Opioid Growth Factor Receptor (OGFR) Antagonists

By “Opioid Growth Factor Receptor (OGFR) antagonist” is meant any molecule that inhibits, suppresses or causes the cessation of at least one OGFR-mediated biological activity such as N-allyl noroxymorphone or a functional derivative thereof.

In some embodiments, an OGFR antagonist may be an OGFR binding antagonist, namely, a molecule that, interferes with, blocks or otherwise prevents the interaction or binding of the met5-ligand (OGF) to the OGFR. Met-5 is derived from the pro-hormone pro-enkephalin (PENK).

An OGFR binding antagonist may compete with the met5-ligand for binding to the OGFR on the surface of the nuclear membrane, thereby interfering with, blocking or otherwise preventing the binding of the met5-ligand to the OGFR, without triggering the downstream signaling that would otherwise be induced by the binding of the met5-ligand to the OGFR. Alternatively, an OGFR binding antagonist may bind to or sequester pro-enkephalin (PENK) or the met5-ligand with sufficient affinity and specificity to substantially interfere with, block or otherwise prevent binding of met5-ligand to the OGFR, thereby inhibiting, suppressing or causing the cessation of at least one OGFR-mediated biological activity. Generally speaking, OGFR binding antagonists may be large molecules (e.g., antibodies) or small molecules (e.g., compounds of a molecular weight of less than 15-kD, 12-kD, 10-kDor even 8-kD), and may be a polypeptide, nucleic acid, or a synthetic small molecule compound. OGFR binding antagonists may be identified with any in vitro assay readily selected by one of skill in the art. For example, OGFR antagonists may be identified using the methods described in Zagon et al., Brain Research Reviews, 2002, 38(3):351-76. Other suitable OGFR antagonists are disclosed in PCT patent application publications Nos. WO2021/011529; WO2022/015364; U.S. patent application publications Nos. 2019-0093109; 2021-0030746; 2021-0228571; 2022-0016312; and U.S. Pat. No. 11,471,454.

In one embodiment, the OGFR binding antagonist may be N-allyl noroxymorphone or a functional derivative thereof, naltrexone or a functional derivative thereof, or a combination thereof.

As used herein, a “functional derivative” refers to a derivative or analog that is structurally and functionally analogous to the originating molecule (e.g., maintains the function of naltrexone or N-allyl noroxymorphone as an OGFR antagonist). N-allyl noroxymorphone and naltrexone analogs can be synthesized using standard synthetic procedures such as those described in March J., Advanced Organic Chemistry, 3rd Ed. (1985). Examples of naltrexone and N-allyl noroxymorphone functional derivatives include salt forms, e.g., N-allyl noroxymorphone hydrochloride dihydrate or naltrexone hydrochloride. Additional examples of naltrexone and N-allyl noroxymorphone functional derivatives suitable for use in the present methods include naltrexone and N-allyl noroxymorphone analogs disclosed in U.S. Patent Application Publication No. 2007/0197573 A1, U.S. Pat. No. 6,713,488, for example.

In another embodiment, an OGFR binding antagonist may be derived from oxymorphone and binds to the OGFR, which includes N-allyl noroxymorphone, naltrexone, nalorphine, naloxonazine, levallorphan, nalmefene, cyprodime, cyclorphan, cyclazocine, oxilorphan, LY113878, MR2266, diprenorphine, WIN 44,441-3, naltindole, or norbinaltorphimine.

In one embodiment, the OGFR binding antagonist may be N-allyl noroxymorphone.

In still another embodiment, an OGFR binding antagonist may be derived from trans-3,4-dimethyl-4-phenylpiperidine and binds to the OGFR, which includes LY99335, LY25506, LY117413, or LY255582.

In another embodiment, an OGFR binding antagonist is derived from the met5-enkephalin or leu-enkephalin peptides, binds to the OGFR, and minimally includes the following amino acid sequences as a means of targeting the OGFR: Tyr-Gly-Gly-Phe-Met (SEQ ID NO: 3) for those derived from met5-enkephalin or Tyr-Gly-Gly-Phe-Leu (SEQ ID NO: 4) for those derived from the leu-enkephalin.

In still another embodiment, an OGFR binding antagonist is derived from the peptide antagonist 101174864 (N,N-diallyl-Tyr-Aib-Aib-Phe-Leu-OH; Aib=aminoisobutyticacid) or somatostatin analog CTP(D-Phe-Cys-Tyr-D-Trp-Lys-Thr-Pen-Thr-NH.sub.2 (SEQ ID NO: 5)).

In other embodiments, the OGFR antagonist, instead of being an OGFR binding antagonist, is a molecule that disrupts the nuclear localization sequence found within OGFR: 251 QSALDYFMFAVRCRHQRRQLVHFAWEHFRPRCKFVWGPQDKLRRFKPSSL (SEQ ID NO: 6).

In still other embodiments, the OGFR antagonist employed in the present methods is a small-hairpin RNA (shRNA) or a small-interfering RNA (siRNA) directed against the OGFR gene and effective in disrupting OGFR gene expression.

The OGFR antagonists described herein may be administered individually or in combination. Suitable combinations include, for example, N-allyl noroxymorphone and naltrexone; N-allyl noroxymorphone and/or naltrexone, in combination with another OGFR binding antagonist or another OGFR antagonist.

### Synergism Between Lanthanum Compounds and N-Allyl Noroxynorphone

Lanthanum compounds and the OGFR antagonist N-allyl noroxymorphone act synergistically to reduce cell proliferation and increase cell death. This is accomplished via the N-allyl noroxymorphone antagonism of the MOR and the OGFR. N-allyl noroxymorphone inhibition of MOR signaling leads to decreased MAPK/PI3K signaling (FIGS. 1A-1B). In parallel, N-allyl noroxymorphone inhibition of OGFR signaling leads to increased p21 expression (FIGS. 1A-1B). Thus, the synergistic decrease in MAPK/PI3K with increased p21 expression leads to decreased cell proliferation (FIGS. 1A-1B). Independently, Lanthanum compounds also inhibit cellular metabolism via TRP channels in mitochondria, as well as the cell's outer membrane. Treatment with a lanthanum compound leads to a decrease in ATP, which in turn reduces cellular activity and further reduces cellular signaling, with many signaling pathways requiring ATP to function (FIGS. 1A-1B).

### Compositions Comprising Lanthanum Compounds

According to one embodiment, described herein is a composition comprising a lanthanum compound and a carrier. In one aspect the carrier is a pharmaceutically acceptable carrier. In some embodiments, the composition may be used for therapeutic methods. In some embodiments, the composition comprising a lanthanum compound is a controlled release formulation.

In some embodiments, the lanthanum compound is an inorganic lanthanum salt or a derivative thereof. In some embodiments the lanthanum compound is lanthanum carbonate.

In some embodiments, the lanthanum compound is present in the composition in an amount of 0.1% to 85% (w/w).

In some embodiments, the lanthanum compound may be at least 0.1% (w/w), or at least 0.5% (w/w), or at least 1% (w/w), or at least 2% (w/w), or at least 5% (w/w), or at least 10% (w/w), or at least 15% (w/w), or at least 20% (w/w), or at least 25% (w/w), or at least 30% (w/w), or at least 35% (w/w), or at least 40% (w/w), or at least 45% (w/w), or at least 50% (w/w), or at least 55% (w/w), or at least 60% (w/w), or at least 65% (w/w), or at least 70% (w/w), or at least 80% (w/w), or at least 85% (w/w) or at least 90% (w/w), or at least 95% (w/w) of the composition.

In some embodiments the concentration of the lanthanum compound in the composition may be between 10-nM to 10-mM.

In some embodiments, the concentration of the lanthanum compound may be at least 10-nM, or at least 50-nM, or at least 100-nM, or at least 200-nM, or at least 300-nM, or at least 400-nM, or at least 500-nM, or at least 600-nM, or at least 700-nM, or at least 800-nM, or at least 900-nM, or at least 1-μM, or at least 5-μM, or at least 10-μM, or at least 50 μM, or at least 100-μM, or at least 200-μM, or at least 250-μM, or at least 300-μM, or at least 350-μM, or at least 400-μM, or at least 4500-μM, or at least 500-μM, or at least 550-μM, or at least 600-μM, or at least 650-μM, or at least 700-μM, or at least 750-μM, or at least 800-μM, or at least 850-μM, or at least 900-μM, or at least 950-μM or at least 1-mM, or at least 2-mM, or at least 3-mM, or at least 4-mM, or at least 5-mM, or at least 5-mM, or at least 7-mM, or at least 8-mM, or at least 9-mM, or at least 10 mM.

In some embodiments, the concentration of the lanthanum compound may be about 10-nM, or about 50-nM, or about 100-nM, or about 200-nM, or about 300-nM, or about 400-nM, or about 500-nM, or about 600-nM, or about 700-nM, or about 800-nM, or about 900-nM, or about 1-μM, or about 5-μM, or about 10-μM, or about 50 μM, or about 100-μM, or about 200-μM, or about 250-μM, or about 300-μM, or about 350-μM, or about 400-μM, or about 4500-μM, or about 500-μM, or about 550-μM, or about 600-μM, or about 650-μM, or about 700-μM, or about 750-μM, or about 800-μM, or about 850-μM, or about 900-μM, or about 950-μM or about 1-mM, or about 2-mM, or about 3-mM, or about 4-mM, or about 5-mM, or about 5-mM, or about 7-mM, or about 8-mM, or about 9-mM, or about 10 mM.

In some embodiments, the concentration of the lanthanum compound may be about 200-nM, about 210-nM, about 220-nM, about 230-nM, about 240-nM, or about 250-nM.

In compositions comprising an OGFR antagonist, in some embodiments, when the OGFR antagonist is N-allyl noroxymorphone, naltrexone or their combination, a concentration of the OGFR antagonist may be at least 0.5-mM, or at least 1-mM, or at least 2-mM, or at least 3-mM, or at least 4-mM, or at least 5-mM, or at least 6-mM, or at least 7-mM, or at least 8-mM, or at least 9-mM, or at least 10-mM, or at least 11-mM, or at least 12-mM, or at least 13-mM, or at least 14-mM, or at least 15-mM, or at least 16-mM, or at least 17-mM, or at least 18-mM, or at least 19-mM, or at least 20-mM, or at least 21-mM, or at least 22-mM, or at least 23-mM.

In some embodiments, the composition may be a liquid formulation.

In some embodiments, the composition may be a percutaneous formulation, i.e., a formulation for a percutaneous mode of administration.

In some embodiments, the composition may be configured to release the lanthanum compound over a period of 24-hours, 48-hours, 72-hours, 96-hours, 120-hours, 144-hours, or 168-hours.

In some embodiments, the formulation possesses a particular level of flowability. In some embodiments, the formulation can pass through a 27-G, 25-G, 23-G, 22-G, 21-G, or 18-G needle.

In some embodiments, the formulation may be such that it does not dissipate from the site of the local administering, which may be, for example, a site of a metastatic cancer lesion, while the lanthanum compound and/or additional therapeutic agent, is being released.

In some embodiments, at least one pharmaceutically acceptable carrier in the composition may be a salt of alginic acid (i.e., an alginate), which may optionally be included in an amount of 5% to 80% (w/w). The alginate may serve as a carrier and also a release rate controlling agent in the composition.

In some embodiments, at least one pharmaceutically acceptable carrier in the composition may include at least one natural gum. Non-limiting examples of natural gums include agar, alginic acid, sodium alginate, carrageenan, gum Arabic, gum ghatti, gum tragacanth, karaya gum, guar gum, locust bean gum, beta-glucan, dammar gum, glucomannan, psyllium seed husks, tara gum, gellan gum and xanthan gum.

In some embodiments, at least one pharmaceutically acceptable carrier in the composition may include gum Arabic, which may optionally be included an amount of 6% to 60% (w/w) and which may serve as a carrier and also a release rate controlling agent in the composition.

In some embodiments, the at least one pharmaceutically acceptable carrier may include at least one iron salt. Suitable iron excipients are disclosed, for example, in U.S. Patent Application No. 2022-0016312, which is incorporated herein by reference in its entirety. In some embodiments, the at least one iron salt may include ferric sulfate, such as Fe(II) or Fe(III) sulfate, which may be present from 0% to 15% (w/w/) and provides stability to the carrier.

In some embodiments, the at least one pharmaceutically acceptable carrier may include at least one titanium salt. Suitable titanium salts are disclosed, for example, titanium oxide. In some embodiments, the at least one iron excipient may include ferric sulfate which may optionally be present from 0% to 15% (w/w) and which may provide color, stability and also contrast when imaging using CT.

In some embodiments, the at least one pharmaceutically acceptable carrier may include at least one sodium phosphate salt. Suitable sodium phosphate salts include sodium phosphate dibasic, sodium phosphate monobasic, and sodium phosphate dibasic dihydrate. In some embodiments, the at least one sodium phosphate salt may include sodium phosphate dibasic which may optionally be present from 0% to 10% (w/w) and which serves a release rate controlling agent.

In some embodiments, the at least one pharmaceutically acceptable carrier may include a sodium alginate, gum Arabic, sodium phosphate dibasic, ferric sulfate, and titanium oxide.

Suitable carriers can be combined with the composition in bead, microsphere or nanoparticle form, and can be made of natural and/or synthetic biocompatible polymers.

Examples of suitable biocompatible polymers include hyaluronic acid, collagen, tricalcium phosphate, chondroitin sulfate, polybutyrate, polylactide, polyglycolide, and lactide/glycolide copolymers, and mixtures or copolymers thereof. Suitable carriers also include on-polymer systems such as carboxylic acids, fatty acids, phospholipids, amino acids, lipids such as sterols, hydrogel release system; silastic system; peptide-based system; implants and the like.

In one embodiment, the carrier is a hygroscopic collagen-based carrier such as a collagen sponge, a collagen scaffold, a powdered collagen, or a collagen-based gelatin hydrogel.

In another embodiment, the carrier is a hydrophilic hydrogel-based carrier (e.g., poly lactic acid, poly glycolic acid), which allows a lanthanum compound infused therein to be released over a period of time.

In another embodiment, the carrier is a carrier composed of a tri-block co-polymer comprising a central block of PLA (poly-(lactic acid) flanked by two blocks of PEG-(poly-(ethylene glycol).

In still another embodiment, the carrier is albumin, a derivative or fragment of albumin that maintains the N-allyl noroxymorphone/morphine binding site located at the interface between the IA and IIA domains, and/or maintains the N-allyl noroxymorphone binding site around tryptophan (Trp)-214, that binds an OGFR antagonist such as N-allyl noroxymorphone or naltrexone or a functional derivative thereof and allows for a slow release of the OGFR antagonist.

In still another embodiment, methyl cellulose, and an inert gel, for example, that binds an active compound and allows for a slow release of the active compound.

In another embodiment, the carrier is a carrier composed of PGA (poly-(glycolic acid)-PLGA (poly-(lactic glycolic acid)) spheres, which can encapsulate a lanthanum compound to provide for immediate, delayed or sustained release.

Suitable carriers can be in bead, microsphere or nanoparticle form, and can be made of natural and/or synthetic biocompatible polymers. Examples of suitable biocompatible polymers include hyaluronic acid, collagen, tricalcium phosphate, chondroitin sulfate, polybutyrate, polylactide, polyglycolide, and lactide/glycolide copolymers, and mixtures or copolymers thereof. Suitable carriers also include on-polymer systems such as carboxylic acids, fatty acids, phospholipids, amino acids, lipids such as sterols, hydrogel release system; silastic system; peptide-based system; implants and the like.

In one embodiment, the carrier is a hygroscopic collagen-based carrier such as a collagen sponge, a collagen scaffold, a powdered collagen, or a collagen-based gelatin hydrogel.

In another embodiment, the carrier is a hydrophilic hydrogel-based carrier (e.g., poly lactic acid, poly glycolic acid), which allows an active compound infused therein to be released over a period of time.

In another embodiment, the carrier is a carrier composed of a tri-block co-polymer comprising a central block of PLA (poly-(lactic acid) flanked by two blocks of PEG-(poly-(ethylene glycol).

In still another embodiment, methyl cellulose, and an inert gel, for example, that binds an active compound and allows for a slow release of the active compound.

In another embodiment, the carrier is a carrier composed of PGA (poly-(glycolic acid)-PLGA (poly-(lactic glycolic acid)) spheres, which can encapsulate an active compound to provide for immediate, delayed or sustained release.

In some embodiments, a pharmaceutical composition disclosed herein comprises one or more “pharmaceutically acceptable carriers,” such as an aqueous carrier, buffer, antioxidants, and/or diluents.

In some embodiments, the acidified saline-based solution exhibits a pH from about 4.5 to about 7.4. In some embodiments, the acidified saline-based solution exhibits a pH from about 5.5 to about 7.4. In some embodiments, the acidified saline-based solution exhibits a pH from about 6.5 to about 7.4.

In some embodiments, the diluent is an ethanol based saline solution. In some embodiments, the ethanol based saline solution comprises from about 1% volume/volume (v/v) ethanol to about 80% v/v ethanol. In some embodiments, the ethanol based saline solution comprises from about 5% volume/volume (v/v) ethanol to about 80% v/v ethanol. In some embodiments, the ethanol based saline solution comprises from about 10% volume/volume (v/v) ethanol to about 80% v/v ethanol. In some embodiments, the ethanol based saline solution comprises from about 15% volume/volume (v/v) ethanol to about 80% v/v ethanol. In some embodiments, the ethanol based saline solution comprises from about 25% volume/volume (v/v) ethanol to about 80% v/v ethanol. In some embodiments, the ethanol based saline solution comprises from about 30% volume/volume (v/v) ethanol to about 80% v/v ethanol. In some embodiments, the ethanol based saline solution comprises from about 35% volume/volume (v/v) ethanol to about 80% v/v ethanol. In some embodiments, the ethanol based saline solution comprises from about 40% volume/volume (v/v) ethanol to about 80% v/v ethanol. In some embodiments, the ethanol based saline solution comprises from about 40% volume/volume (v/v) ethanol to about 70% v/v ethanol.

In some embodiments, the ethanol based saline solution comprises about 1% volume/volume (v/v) ethanol. In some embodiments, the ethanol based saline solution comprises about 2% volume/volume (v/v) ethanol. In some embodiments, the ethanol based saline solution comprises about 3% volume/volume (v/v) ethanol. In some embodiments, the ethanol based saline solution comprises about 4% volume/volume (v/v) ethanol. In some embodiments, the ethanol based saline solution comprises about 5% volume/volume (v/v) ethanol. In some embodiments, the ethanol based saline solution comprises about 6% volume/volume (v/v) ethanol. In some embodiments, the ethanol based saline solution comprises about 7% volume/volume (v/v) ethanol. In some embodiments, the ethanol based saline solution comprises about 8% volume/volume (v/v) ethanol. In some embodiments, the ethanol based saline solution comprises about 9% volume/volume (v/v) ethanol. In some embodiments, the ethanol based saline solution comprises about 10% volume/volume (v/v) ethanol. In some embodiments, the ethanol based saline solution comprises about 20% volume/volume (v/v) ethanol. In some embodiments, the ethanol based saline solution comprises about 30% volume/volume (v/v) ethanol. In some embodiments, the ethanol based saline solution comprises about 40% volume/volume (v/v) ethanol. In some embodiments, the ethanol based saline solution comprises about 50% volume/volume (v/v) ethanol. In some embodiments, the ethanol based saline solution comprises about 60% volume/volume (v/v) ethanol. In some embodiments, the ethanol based saline solution comprises about 70% volume/volume (v/v) ethanol. In some embodiments, the ethanol based saline solution comprises about 80% volume/volume (v/v) ethanol.

In some embodiments, the ethanol based saline solution comprises a phosphate buffered saline solution, a borate buffered saline solution, a Tris buffered saline solution, or a carbonate buffered saline solution.

In some embodiments, the saline solution comprises a salt and water. In some embodiments, the salt of the saline solution comprises sodium chloride or potassium chloride. In some embodiments, the saline solution comprises from about 0.7% w/w salt to about 1.5% w/w salt. In some embodiments, the saline solution comprises about 0.7% salt. In some embodiments, the saline solution comprises about 0.8% salt. In some embodiments, the saline solution comprises about 0.9% salt. In some embodiments, the saline solution comprises about 1.0% salt. In some embodiments, the saline solution comprises about 1.1% salt. In some embodiments, the saline solution comprises about 1.2% salt. In some embodiments, the saline solution comprises about 1.3% salt. In some embodiments, the saline solution comprises about 1.4% salt. In some embodiments, the saline solution comprises about 1.5% salt.

The lanthanum compound may be combined or coordinately administered with a suitable carrier or vehicle depending on the route of administration. The term “pharmaceutically acceptable carrier” refers to a carrier that is conventionally used in the art to facilitate the storage, administration, and/or the healing effect of an active agent of a pharmaceutical composition.

A water-containing liquid carrier can comprise pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories can be found in the U.S. Pharmacopeia National Formulary, 1857-1859, and (1990). Some examples of the materials which can serve as pharmaceutically acceptable carriers are sugars, such as lactose, glucose and sucrose; cyclodextrins, including alpha-cyclodextrin, beta-cyclodextrin, and gamma-cyclodextrin; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents.

In some embodiments, the pharmaceutical composition comprises preservatives and antioxidants. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art. Examples of filling agents include lactose monohydrate, lactose anhydrous, and various starches; examples of binding agents include various celluloses and cross-linked polyvinylpyrrolidone, microcrystalline cellulose such as Avicel™, PH101 microcrystalline cellulose and/or Avicel™, PH102 microcrystalline cellulose, and silicified microcrystalline cellulose such as ProSolv SMCC™. Suitable lubricants, including agents that act on the flow-ability of the powder to be compressed, may include colloidal silicon dioxide such as Aerosil® 200 (colloidal silicon dioxide), talc, stearic acid, magnesium stearate, calcium stearate, and silica gel. Examples of sweeteners may include any natural or artificial sweetener, such as sucrose, xylitol, sodium saccharin, cyclamate, aspartame, and acesulfame. Examples of flavoring agents are Monoammonium Glycyrrhizinate such as Magnasweet™ (a flavoring composition containing Monoammonium Glycyrrhizinate and trademark of MAFCO), bubble gum flavor, and fruit flavors, and the like. Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Any pharmaceutical formulation used for therapeutic administration can be sterile. Sterility is readily accomplished by for example filtration through sterile filtration membranes (e.g., 0.2 micron membranes). Any pharmaceutically acceptable sterility method can be used in the formulation.

The pharmaceutical composition or formulation comprising a lanthanum compound will be formulated and dosed in a fashion consistent with good medical practice, taking into account the clinical condition of the individual patient, the method of administration, the scheduling of administration, and other factors known to those in the art.

A variety of administration routes are available. The pharmaceutical composition or formulation of the invention may be practiced using any mode of administration that is medically acceptable, meaning any mode that produces effective levels of the active ingredients without causing clinically unacceptable adverse effects. Accordingly, the pharmaceutical compositions or formulations can be administered to a subject parenterally, intraperitoneally, transdermally, intramuscularly, intratumorally, subcutaneously, intra-adiposally, intra-articularly, or intrathecally.

Modes of administration include topical, intradermal, or parenteral routes. The term “parenteral” includes subcutaneous, percutaneous, intravenous, intramuscular, or infusion.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's solution or fixed 25 oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

In some embodiments, the controlled release formulation may be administered locally to a site (e.g., intratumorally, peritumorally, or perilesionally), such as a site of a metastatic cancer lesion, in a subject via percutaneous injection, any minimally invasive procedure, or via laparoscopy. The controlled release formulation may be such that it does not dissipate from the site of percutaneous injection. The act of local administering such as percutaneous injection to the site, such as the site of a metastatic cancer lesion, may be repeated for as long is tolerable to the patient and effective for reducing tumor, including but not limited to at least one additional time, or at least two time, or at least three times, or at least four times.

In some embodiments, the controlled release formulation is administered locally at a local dosage of the lanthanum-compound, of about 10-nM to about 10-mM. In some embodiments, the local dosage may be about 10-nM, 50-nM, 100-nM, 200-nM, 300-nM, 400-nM, 500-nM, 600-nM, 700-nM, 800-nM, 900-nM, 1-μM, 5-μM, 10-μM, 50 μM, 100-μM, 200-μM, 250-μM 300-μM, 350-μM, 400-μM, 4500-μM, 500-μM, 550-μM, 600-μM, 650-μM, 700-μM, 750-μM, 800-μM, 850-μM, 900-μM, 950-μM 1-mM, 2-mM, 3-mM, 4-mM, 5-mM, 6-mM, 7-mM, 8-mM, 9-mM, or 10 mM.

In some embodiments, the lanthanum is administered with a carrier, and the carrier volume may be from about 0.1-cubic centimeter (cc) to about 20-cc, from about 0.25-cc to about 15-cc, from about 0.5-cc to about 10-cc, from about 1-cc to about 10-cc, from about 2-cc to about 10-cc. The corresponding amount of the active compound in the carrier may be from about 0.2 mg per cc, 0.5 mg per cc, 1.0 mg per cc, 2.0 mg per cc, 3.0 mg per cc, 4.0 mg per cc, 5.0 mg per cc, 6.0 mg per cc, 7.0 mg per cc, 8.0 mg per cc, 9.0 mg per cc, and/or 10.0 mg per cc.

The methods and compositions herein may be provided in the form of a kit. A “kit” is herein defined as a package and containing several individual parts that show a complementary effect when applied together. In this aspect, the effect achieved by a kit and the pharmaceutical composition are similar. The kit may optionally include instructions for using the pharmaceutical compositions.

### Cancer

In some embodiments, the composition may be used for treating a cancer.

Cancer may refer to a condition in which abnormal cells divide without control and can invade nearby tissues.

In some embodiments, the cancer may be osteosarcoma, breast cancer, lung cancer, prostate cancer, or brain cancer. In some embodiments, the cancer may be a carcinoma, i.e., a cancer that arises from tissue that functions to line, cover, or act as a barrier for internal organs, such as the skin or breast epithelium. In some embodiments, the cancer may be sarcoma, i.e., a connective tissue cancer that can begin in bone, cartilage, fat, muscle, or other connective or supportive tissue. In some embodiments, the cancer may be a hematological cancer, i.e., a blood cancer that starts in or includes blood-forming tissue, such as the bone marrow, and causes large numbers of abnormal blood cells to be produced and enter the blood. In some embodiments, the cancer is a lymphoma or multiple myeloma, i.e., a cancer that begin in the cells of the immune system. In some embodiments, the cancer may be a central nervous system cancer, i.e., a cancer that begin in a tissue of the brain and/or spinal cord. In some embodiments, the cancer is one or more of pancreatic cancer, renal cancer, small cell lung cancer, brain cancer, neural cancer, bone cancer, lymphoma, myeloma, gastrointestinal tract cancer, uterine cancer, breast cancer, leukemia, liver cancer, prostate cancer, skin cancer, and melanoma. In some embodiments, the cancer is soft tissue non-osteogenic sarcomas, chondrosarcoma, fibrosarcoma, or synovial sarcoma. In some embodiments, the cancer is specifically, a basal cell carcinoma, melanoma, thyroid adenocarcinomas, glial blastoma, pituitary tumors, oligodendrocytoma, bladder carcinoma, triple negative breast carcinoma, breast carcinoma, non-small cell lung carcinoma, small cell lung carcinoma, prostate carcinoma, neuroblastoma, or astrocytoma.

In some embodiments, the cancer is a breast cancer.

In some embodiments, the cancer is a sarcoma of bone, in the appendicular skeleton, the axial skeleton, and the skull, which includes non-specific bone sarcomas, osteosarcoma, osteogenic sarcomas, Ewing's sarcoma, or benign bone tumors.

In some embodiments, a composition comprising a lanthanum compound may be administered locally to a site of cancerous lesion (e.g., intratumorally, peritumorally or perilesionally), which may be, for example, a site of a primary or metastatic cancer lesion.

In some embodiments, a composition comprising a lanthanum compound may be administered systemically, delivered via normal systemic administration routes, to treat a cancerous lesion, for example a primary or metastatic lesion. Normal routes of systemic administration include oral, intravenous, intraperitoneal, subdural, or intramuscular.

In some embodiments a therapeutically effective amount of a composition comprising a lanthanum compound may be administered locally to a site of cancerous lesion.

In some embodiments, a composition comprising a lanthanum compound may be contacted in vivo or in vitro with a cancer cell. In some embodiments the cancer cell is a MCF7 or BT474 cell.

### Combination Therapy

The present disclosure contemplates the use of the lanthanum compounds disclosed herein alone or in combination with one or more additional therapy. Each additional therapy can be a therapeutic agent or another treatment modality. In embodiments comprising one or more additional therapeutic agents, each agent may target a different, but complementary, mechanism of action.

In embodiments comprising one or more additional treatment modality, the compound can be administered before, after or during treatment with the additional treatment modality. In embodiments comprising one or more additional therapeutic agents, the therapeutic agents used in such combination therapy can be formulated as a single composition or as separate compositions. If administered separately, each therapeutic agent in the combination can be given at or around the same time, or at different times. Furthermore, the therapeutic agents are administered “in combination” even if they have different forms of administration (e.g., oral capsule and intravenous), they are given at different dosing intervals, one therapeutic agent is given at a constant dosing regimen while another is titrated up, titrated down or discontinued, or each therapeutic agent in the combination is independently titrated up, titrated down, increased or decreased in dosage, or discontinued and/or resumed during a subject's course of therapy. If the combination is formulated as separate compositions, in some embodiments, the separate compositions are provided together in a kit.

### EXAMPLES

Decreased Metabolic Activity after Treatment with Lanthanum

BT474 cancer cells were treated with 50-mg/mL of lanthanum carbonate, allowed to incubate overnight, and then assayed with 4,5-dimethylthiazol-2-yl)-2,5-diphenyltetrazolium bromide. The 4,5-dimethylthiazol-2-yl)-2,5-diphenyltetrazolium bromide was allowed to incubate with the cells for 2-hours, and then imaged using a Nikon Eclipse Ni microscope. The 4,5-dimethylthiazol-2-yl)-2,5-diphenyltetrazolium bromide reacts with NAPDH located on the mitochondrial membrane and is converted into a purple crystal only if the mitochondria are active. Thus, 4,5-dimethylthiazol-2-yl)-2,5-diphenyltetrazolium bromide can be used to assess normal cellular metabolism. The images show that a single 50-mg/mL dose of lanthanum carbonate produced a significant decrease in the purple color, indicating a significant decrease in cellular metabolism. See FIG. 2.

### IC50 of Lanthanum Carbonate

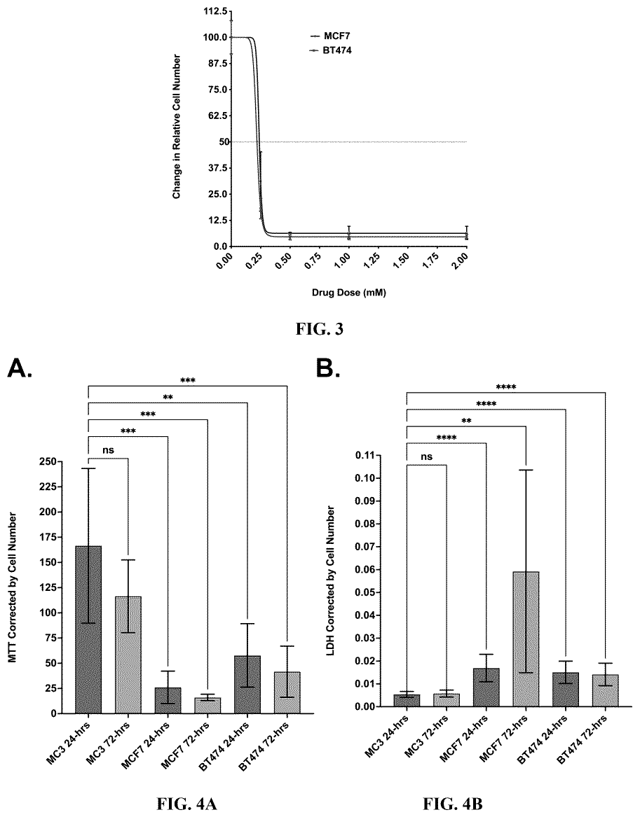

Applicant exposed different concentrations of lanthanum carbonate to MCF7 or BT474 cancer cells to determine the IC50 for lanthanum carbonate. The MCF7 IC50 is 237.6-μg and the BT474 IC50 is 217.5-μg. These data show a high level of sensitivity to lanthanum carbonate treatment. See FIG. 3.

### MTT and LDH Assays

Cells were treated with a lanthanum carbonate hydrogel and tested for its effects on cell proliferation and cell death using the MTT and LDH (lactate dehydrogenase) assays, corrected for cell number to allow for the comparison between different cell-lines. The MC3T3-E1 (MC3) cell-line was used as non-tumor cell control.

MTT: The MTT assay provides a measurement of cell metabolic activity. For example, cell cultures with lower MTT relative to the control have lower metabolic activity. Following treatment with the lanthanum hydrogel (Table 1), no significant change in MC3 cell number at 24- or 72-hours (FIG. 4A) was observed. Relative to the MTT values for the MC3 cells at 24-hours, in MCF7 breast cancer cell cultures there was a significant 6.41-fold decrease in cell number at 24-hours (p<0.0003) and a 10.41-fold decrease in cell number at 72-hours (p<0.0001). Similarly, when compared to the MTT values for the MC3 cells at 24-hours, the MTT values for the BT474 breast cancer cells were significantly decreased 2.89-fold (p<0.0019) at 24-hours and 4-fold at 72-hours (p<0.0005).

TABLE 1
Lanthanum hydrogel composition.

| | Dry Mass |
|---|---|
| Master Mix Composition | w/w (%) |
| Carrier | |
| Lanthanum carbonate | 26.53 |
| Sodium Alginate | 53.06 |
| Gum Arabic | 11.87 |
| Sodium phosphate | 2.97 |
| Titanium oxide | 3.71 |
| Iron (III) sulfate hydrate [Fe2(SO4)3] | 1.86 |
| Total | 100.0 |
| Diluent Solution [Saline + Citrate + Acetic Acid] | |
| 0.9 wt % Saline Solution [0.95-mL/mL] | 90.2% |
| 0.9 wt % Citric acid monohydrate [C6H8O7* H2O] | 0.9% |
| [8.82-mg/mL] | |
| 5% Acetic acid [0.05-mL/mL] | 5.0% |
| 4.8% Ethanol | 4.8% |
| 10-mM Naloxone HCL [4-mg/mL] | |
| Total Diluent Solution [mL] | 1.000 |
| Carrier | |
| Lanthanum carbonate | 26.53 |
| Sodium Alginate | 53.06 |
| Gum Arabic | 11.87 |
| Sodium phosphate | 2.97 |
| Titanium oxide | 3.71 |
| Iron (III) sulfate hydrate [Fe2(SO4)3] | 1.86 |
| Total | 100.0 |
| Diluent Solution [Saline + Citrate + Acetic Acid] | |
| 0.9 wt % Saline Solution [0.95-mL/mL] | 90.2% |
| 0.9 wt % Citric acid monohydrate [C6H8O7* H2O] | 0.9% |
| [8.82-mg/mL] | |
| 5% Acetic acid [0.05-mL/mL] | 5.0% |
| 4.8% Ethanol | 4.8% |
| 10-mM Naloxone HCL [4-mg/mL] | |
| Total Diluent Solution [mL] | 1.000 |

LDH: The LDH assay is used to measure cell death. For example, cell cultures with lower LDH relative to the control have lower cell death. After treatment with the lanthanum containing hydrogel no significant change in MC3 cell death at 24- or 72-hours (FIG. 4B) was observed. However, relative to the LDH values for the MC3 cells at 24-hours, in MCF7 cultures there was a significant 3.15-fold increase in LDH (p<0.0001) at 24-hours and a 11-fold increase in LDH (p<0.0044) at 72-hours. For BT474 cultures there was a significant 2.8-fold increase in LDH (p<0.0001) at 24-hours and 2.62-fold at 72-hours.

### EMBODIMENTS

Exemplary embodiments include:
1. A composition for treating cancer, comprising a lanthanum compound and a pharmaceutically acceptable carrier.
2. The composition of embodiment 1, wherein the lanthanum compound is selected from the group comprising: lanthanum salt, lanthanum carbonate, lanthanum chloride, or a functional derivative thereof.
3. The composition of embodiment 1 or 2, wherein the lanthanum is included in an amount of about 1% to 80% (w/w).
4. The composition of any one of embodiments 1-3, wherein the lanthanum is included in a concentration of about 10-nM to about 10-mM.
5. The composition of any one of embodiments 1-4, wherein the lanthanum is included in a concentration of at least 10-nM, or at least 50-nM, or at least 100-nM, or at least 200-nM, or at least 300-nM, or at least 400-nM, or at least 500-nM, or at least 600-nM, or at least 700-nM, or at least 800-nM, or at least 900-nM, or at least 1-μM, or at least 5-μM, or at least 10-μM, or at least 50 μM, or at least 100-μM, or at least 200-μM, or at least 250-μM, or at least 300-μM, or at least 350-μM, or at least 400-μM, or at least 4500-μM, or at least 500-μM, or at least 550-μM, or at least 600-μM, or at least 650-μM, or at least 700-μM, or at least 750-μM, or at least 800-μM, or at least 850-μM, or at least 900-μM, or at least 950-μM or at least 1-mM, or at least 2-mM, or at least 3-mM, or at least 4-mM, or at least 5-mM, or at least 5-mM, or at least 7-mM, or at least 8-mM, or at least 9-mM, or at least 10 mM.
6. The composition of any one of embodiments 1-5, wherein the lanthanum is included in a concentration of about 200-nM, about 210-nM, about 220-nM, about 230-nM, about 240-nM, or about 250-nM.
7. The composition of any of embodiments 1-6, wherein the pharmaceutically acceptable carrier is a hydrogel-forming polymer.
8. The composition of embodiment 7, wherein the hydrogel-forming polymer is selected from the group comprising: poly lactic acid, poly glycolic acid, alginate, agarose, collagen, and gelatin.
9. The composition of embodiment 8, wherein the hydrogel-forming polymer is alginate, optionally wherein the alginate is sodium alginate.
10. The composition of any of embodiments 1-9, further comprising an additional therapeutic agent.
11. The composition embodiment 11, wherein the additional therapeutic agent is an opioid growth factor receptor (OGFR) antagonist.
12. The composition of embodiment 8, wherein the OGFR antagonist is selected from the group comprising: N-allyl noroxymorphone, naltrexone, a functional derivative or an analogue thereof, and a combination thereof.
13. A method to inhibit or reduce the growth of cancer cell, comprising contacting the cell in vivo or in vitro with the composition of any one of embodiments 1-12.
14. A method to decrease cell proliferation and protein synthesis in a cancer cell, comprising contacting the cell in vivo or in vitro with the composition of any one of embodiments 1-12.
15. The method of any of embodiment 13 or 14, further comprising contacting the cancer cell with an additional therapeutic agent.
16. The method of embodiment 15, wherein the additional therapeutic agent is an OGFR antagonist.
17. The method of embodiment 16, wherein the OGFR antagonist is selected from the group comprising: N-allyl noroxymorphone, naltrexone, a functional derivative or an analogue thereof, and a combination thereof.
18. A method to inhibit the growth of a cancer and/or increase the survival rate of a subject in need, comprising administering to the subject in need an effective amount of the composition of any one of embodiments 1-12.
19. The method of embodiment 18, further comprising administering to the subject an additional therapeutic agent, wherein the additional therapeutic agent is applied consecutively or concurrently to the lanthanum composition.
20. The method of embodiment 19, wherein the additional therapeutic agent is an OGFR antagonist.
21. The method of embodiment 20, wherein the OGFR antagonist is selected from the group comprising: N-allyl noroxymorphone, naltrexone, a functional derivative or an analogue thereof, and a combination thereof.
22. The method of any of embodiments 18-21, wherein the cancer is a solid tumor.
23. The method of any of embodiments 13-21, wherein the cancer is selected from the group comprising breast cancer, lung cancer, prostate cancer, and glioblastoma.
24. The method of embodiment 23, wherein the cancer is breast cancer.

### EQUIVALENTS

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

What is claimed is:

1. A composition for treating cancer, comprising a lanthanum compound and a pharmaceutically acceptable carrier.

2. The composition of claim 1, wherein the lanthanum compound is selected from the group comprising: lanthanum salt, lanthanum carbonate, lanthanum chloride, or a functional derivative thereof.

3. The composition of claim 1, wherein the lanthanum is included in an amount of about 1% to 80% (w/w).

4. The composition of claim 1, wherein the lanthanum is included in a concentration of about 10-nM to about 10-mM.

5. The composition of claim 1, wherein the lanthanum is included in a concentration of at least 10-nM, or at least 50-nM, or at least 100-nM, or at least 200-nM, or at least 300-nM, or at least 400-nM, or at least 500-nM, or at least 600-nM, or at least 700-nM, or at least 800-nM, or at least 900-nM, or at least 1-μM, or at least 5-μM, or at least 10-μM, or at least 50 μM, or at least 100-μM, or at least 200-μM, or at least 250-μM, or at least 300-μM, or at least 350-μM, or at least 400-μM, or at least 4500-μM, or at least 500-μM, or at least 550-μM, or at least 600-μM, or at least 650-μM, or at least 700-μM, or at least 750-μM, or at least 800-μM, or at least 850-μM, or at least 900-μM, or at least 950-μM or at least 1-mM, or at least 2-mM, or at least 3-mM, or at least 4-mM, or at least 5-mM, or at least 5-mM, or at least 7-mM, or at least 8-mM, or at least 9-mM, or at least 10 mM.

6. The composition of claim 1, wherein the lanthanum is included in a concentration of about 200-nM, about 210-nM, about 220-nM, about 230-nM, about 240-nM, or about 250-nM.

7. The composition of claim 1, wherein the pharmaceutically acceptable carrier is a hydrogel-forming polymer.

8. The composition of claim 7, wherein the hydrogel-forming polymer is selected from the group comprising: poly lactic acid, poly glycolic acid, alginate, agarose, collagen, and gelatin.

9. The composition of claim 8, wherein the hydrogel-forming polymer is alginate, optionally wherein the alginate is sodium alginate.

10. The composition of claim 1, further comprising an additional therapeutic agent.

11. The composition of claim 10, wherein the additional therapeutic agent is an opioid growth factor receptor (OGFR) antagonist.

12. The composition of claim 8, wherein the OGFR antagonist is selected from the group comprising: N-allyl noroxymorphone, naltrexone, a functional derivative or an analogue thereof, and a combination thereof.

13. A method to inhibit or reduce the growth of cancer cell, comprising contacting the cell in vivo or in vitro with the composition of claim 1.

14. A method to decrease cell proliferation and protein synthesis in a cancer cell, comprising contacting the cell in vivo or in vitro with the composition of claim 1.

15. The method of claim 13, further comprising contacting the cancer cell with an additional therapeutic agent.

16. The method of claim 15, wherein the additional therapeutic agent is an OGFR antagonist.

17. The method of claim 16, wherein the OGFR antagonist is selected from the group comprising: N-allyl noroxymorphone, naltrexone, a functional derivative or an analogue thereof, and a combination thereof.

18. A method to inhibit the growth of a cancer and/or increase the survival rate of a subject in need, comprising administering to the subject in need an effective amount of the composition of claim 1.

19. The method of claim 18, further comprising administering to the subject an additional therapeutic agent, wherein the additional therapeutic agent is applied consecutively or concurrently to the lanthanum composition.

20. The method of claim 19, wherein the additional therapeutic agent is an OGFR antagonist.

21. The method of claim 20, wherein the OGFR antagonist is selected from the group comprising: N-allyl noroxymorphone, naltrexone, a functional derivative or an analogue thereof, and a combination thereof.

22. The method of claim 18, wherein the cancer is a solid tumor.

23. The method of claim 18, wherein the cancer is selected from the group comprising breast cancer, lung cancer, prostate cancer, and glioblastoma.

24. The method of claim 23, wherein the cancer is breast cancer.


SEQUENCE LISTING (6 sequences)

SEQ ID NO: 1 - protein, 8 aa, synthetic construct
  SITE 3: note = 2-aminobutyric acid
  SITE 8: note = Amidated p-Fluorophenylalanine
AAXRRLIF

SEQ ID NO: 2 - protein, 8 aa, synthetic construct
  SITE 8: note = Amidated residue
HAKRRLIF

SEQ ID NO: 3 - protein, 5 aa, synthetic construct
YGGFM

SEQ ID NO: 4 - protein, 5 aa, synthetic construct
YGGFL

SEQ ID NO: 5 - protein, 8 aa, synthetic construct
  SITE 1: note = D-amino acid
  SITE 4: note = D-amino acid
  SITE 7: note = Penicillamine
  SITE 8: note = Amidated residue
FCYWKTXT

SEQ ID NO: 6 - protein, 50 aa, synthetic construct
QSALDYFMFAVRCRHQRRQLVHFAWEHFRPRCKFVWGPQDKLRRFKPSSL